(19) United States
(12) Patent Application Publication
(10) Pub. No.: US 2006/0229492 A1
(43) Pub. Date: Oct. 12, 2006

(54) MATERIALS AND METHODS FOR IN SITU FORMATION OF A HEART CONSTRAINER

(75) Inventors: Mark Gelfand, New York, NY (US); Howard Levin, Teaneck, NJ (US); Ary Chernomorsky, Walnut Creek, CA (US)

Correspondence Address: NIXON & VANDERHYE, PC, 901 NORTH GLEBE ROAD, 11TH FLOOR, ARLINGTON, VA, 22203, US

(73) Assignee: G & L CONSULTING LLC, New York, NY (US)

(21) Appl. No.: 11/279,026

(22) Filed: Apr. 7, 2006

Related U.S. Application Data
(60) Provisional application No. 60/669,355, filed on Apr. 8, 2005.

(51) Int. Cl. A61F 2/00

(52) U.S. Cl. 600/37; 424/422

(57) ABSTRACT

A method to constrain a heart including the steps of: injecting a biopolymer into a pericardial space of the heart; inducing intramolecular or intermolecular interactions in the biopolymer in the pericardial space to modify physical properties of the biopolymer in the pericardial space, and constraining the heart with the modified biopolymer in the pericardial space.

The benefit is claimed of U.S. Provisional Patent Application No. 60/669,355, filed Apr. 8, 2005, which is incorporated by reference.

### BACKGROUND

This invention relates to methods of constraining the heart using implantable devices and methods of fabricating them. In particular, the device is one for forming a heart constrainer in the pericardial sac surrounding the heart to assist in the treatment of heart failure (HF) and expansion of acute myocardial infarction (MI). The device, generically, is an injectable substance placed into the pericardium utilizing a delivery system. The injectable substance is formulated in such a way that upon placement into the pericardial space, it tends to form a polymeric network or matrix, acting as a constraining jacket. There are certain physical and chemical mechanisms coupled with device deployment that induce polymer network formation via intramolecular interactions such as: physical and chemical crosslinking, gelation, and photopolymerization just to mention a few. The nature of the constrainer once it is fully fabricated in situ is that it tends not to allow the heart to expand further with time. The device is preferably placed into the pericardial space using percutaneous or minimally invasive surgical techniques.

### SUMMARY

Multiple animal studies and limited human clinical trials have established benefit of constraining the heart in case of MI and CHF. The purpose of a cardiac constraint is the reduction of the myocardial stress and ventricular dilation. The existing methods and devices for heart constraining are represented by surgical implantation of prefabricated constraining jackets made out of metal or polymer.

A novel treatment procedure, device for the implementation of this procedure, and methods of fabrication of the device has been invented for clinical use. Constraining of the heart is achieved by fabrication of the heart constraining implant in the pericardial space using various injectable synthetic and biopolymers and an array of physical and chemical methods inducing intramolecular interactions resulting in polymerization, crosslinking, and gelation in situ.

An embodiment of a in situ fabricated heart constraining jacket comprises: (i) a cannula or a catheter communicating with the pericardial space, (ii) an external delivery system for containment, conditioning, mixing, and transportation of the principal components of injectable heart implant to the cannula or the catheter in controlled manner, (iii) an injectable substance or combination of substances and/or agents capable of formation of a polymer network acting as a heart constrainer, and (iv) methods for the polymer network formation and stabilization. For example, an injection of crosslinked silicone gel to the pericardial space and induce increase the degree of the crosslinking even further using external radiation and/or slight temperature elevation to form an polymer matrix around heart, acting as a heart constrainer.

The treatment method may include the following steps: (i) placement and securing of the cannula for the injection of the implant's components into the pericardial space, (ii) connection of the delivery system containing constrainer fabricating components to the cannula or the catheter, (iii) preparing, conditioning and mixing components of injectable constrainer, (iv) controlled injection into the pericardial space, (iv) induction of the polymer network formation and stabilization,(v) extraction of the cannula, and (vi) sealing of the transpericardial incision.

The treatment method may include several related aspects. In one aspect, the invention is a treatment procedure for myocardial infarction (MI) and chronic heart failure (CHF). In the other aspect, the method includes fabricating the injectable heart constrainer and deploying the constrainer throughout the entire pericardial space to provide the treatment. The method may be used with a procedure kit including component(s) of an injectable implant in the form of solutions, suspensions, emulsions or gels; a means for administering injectable components into the pericardial space of the patient; and a means for forming a polymer network or matrix, acting as a heart constrainer.

### SUMMARY OF THE DRAWINGS

A preferred embodiment and best mode of the invention is illustrated in the attached drawings that are described as follows:

FIGS. 1A, 1B, 1C, and 1D illustrate a concept of the in situ formation of the polymer network or matrix acting as a heart constrainer. FIG. 1A shows an anatomical details of the heart in partial cross-section. FIG. 1B shows the distal end of the delivery system inserted into the pericardial space during deployment of the injectable implant. FIG. 1C shows a cross-sectional diagram of a portion of the heart post implantation with heart constrainer been formed in pericardial space.

FIGS. 1D and 1E are front and side views that illustrate an initial phase of the treatment procedure of a patient using minimally invasive insertion of the cannula through the subxiphoidal incision into pericardial space. FIG. 1D shows a chest of a person and the principal anatomical structures of heart region.

FIG. 1F is a schematic view of a heart and catheter that illustrates the percutaneous treatment procedure of a patient using minimally invasive insertion of the transvascular catheter. FIGS. 1G and 1H show a cross-sectional diagram of a portion of the heart with a close up of the distal end of the transvascular catheter placed

FIGS. 2A to 2G illustrate the general view and the details of a catheter delivery system for the placement of an injectable implant into the pericardial sac of the heart.

FIG. 2A illustrates the perspective view of the delivery system for the placement of an injectable implant into the pericardial sac of the heart.

FIGS. 2B, D and F are side views of various distal end configurations.

FIGS. 2C, E and G are end views of various distal end configurations.

FIGS. 3A, B, C, D, E, F, G and H are perspective views showing a catheter and heart in partial cross-sectional to illustrate the portion of the heart where the distal tip of the delivery system is embedded in to pericardial sac so the heart constrainer in injected.

FIGS. 4 A, B and C are cross-sectional diagrams of distal end of the delivery system that illustrate the induction mechanisms for the heart constrainer in situ fabrication.

FIGS. 5A to 5D are schematic diagrams showing various methods for formations of hydrogels.

### DETAILED DESCRIPTION OF THE INVENTION

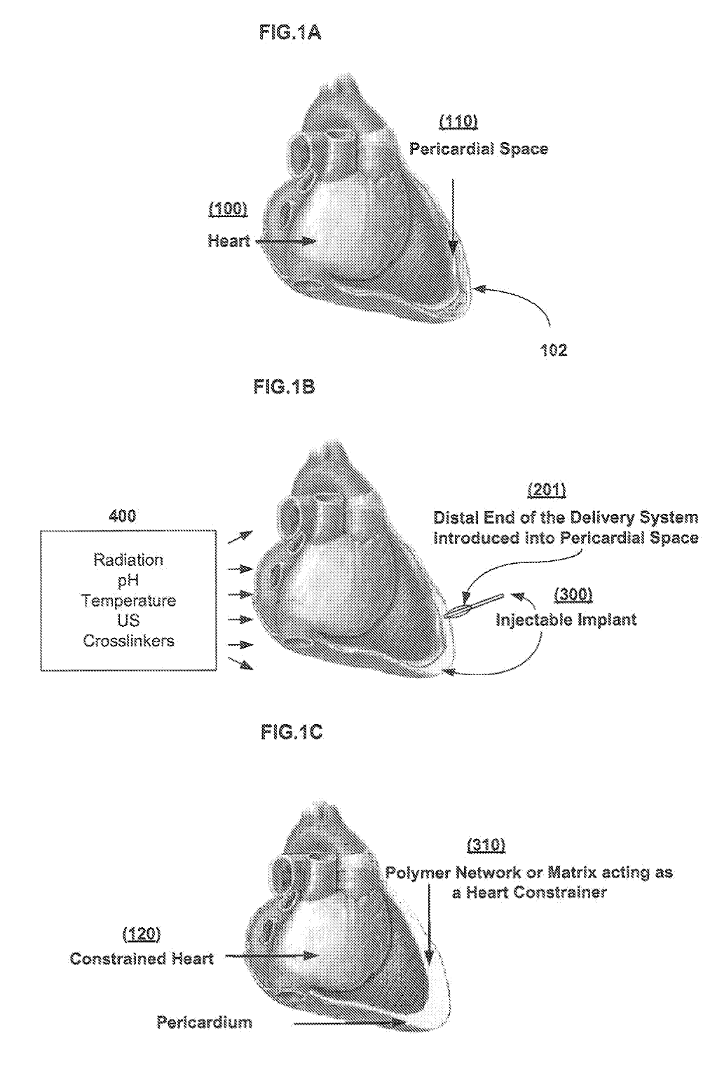

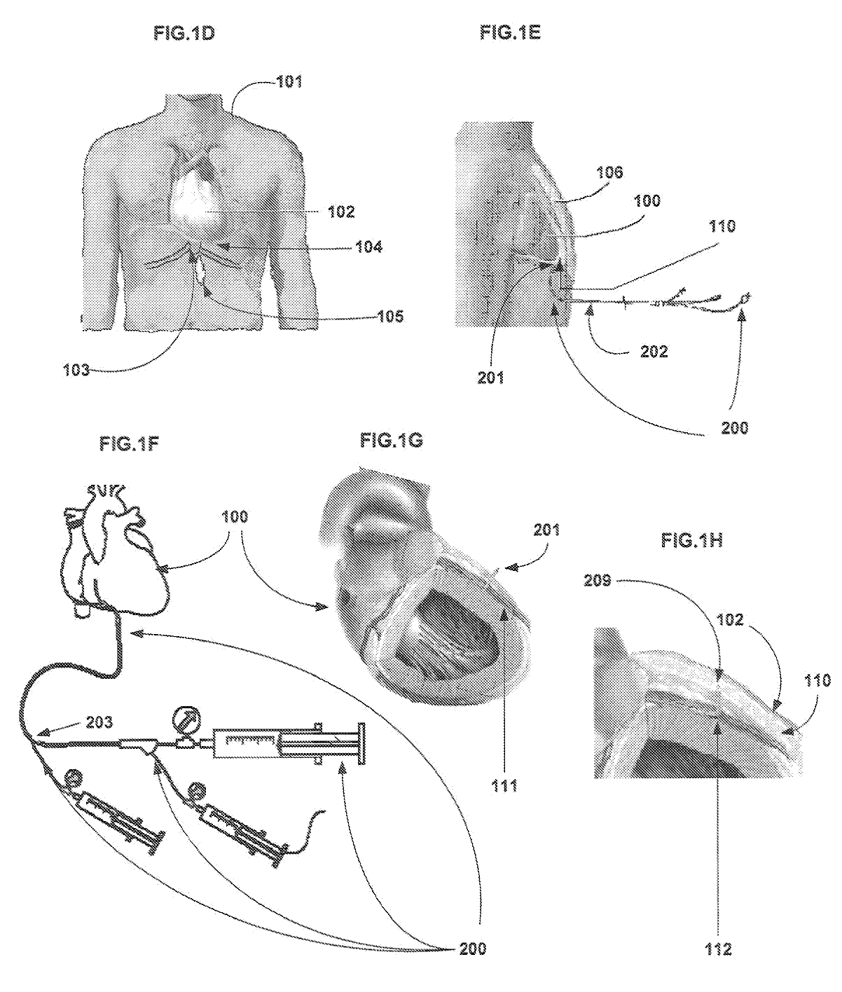

FIGS. 1A, B and C illustrate the concept of in situ heart constrainer fabrication. A mammal heart 100 is surrounded by a membrane called pericardium 102. The space between the outer surface of the heart, also called epicardium and the inner surface of the pericardium is referred to as a pericardial space 110 or pericardial sac. The constrainer in the form of injectable implant 300 is placed into the pericardium utilizing a delivery system. The distal tip of the delivery system 201 has an opening and is in fluid communication with the pericardial (also called interpericardial) space 110. The proximal end of the delivery system 200 containing the components of the implantable PHC, such as sterile crosslinked bovine collagen gel dispersed with PMMA (polymethyl methacrylate) beads.

Upon placement into the pericardial space, the injectable implant fills the pericardial sac 110 and forms a polymeric network or matrix 310 acting as a constraining jacket. There are certain physical and chemical mechanisms 400 (such as heat, light, radiation, pH, crosslinking agents) coupled with implant deployment that induce polymer network formation in situ. Fully fabricated in situ and dispersed throughout pericardial sac matrix prevents constrained heart 120 to expand further with time.

FIGS. 1D and 1E illustrate surgical minimally invasive approach for the treatment of a patient 101 with the system 200 for placement injectable implant into the pericardial sac of the heart and fabrication of polymer heart constrainer (PHC). The distal end 201 of delivery system 200 is partially inserted into the pericardial sac of the heart 100. Cannula (catheter) 202 crosses the patient's skin in the xiphoid area 103 via subxiphoidal incision 105. The diaphragm 104 is incised during surgery down to the pericardial surface. Through this incision 105, the pericardium 102 may be easily visualized and a small incision or a puncture is made in pericardium to accommodate a cannula insertion. The distal tip of the cannula 201 has an opening and is in fluid communication with the pericardial (also called interpericardial) space 110. The proximal end of the cannula 202 is connected to the delivery system 200 containing the components of the implantable PHC, such as sterile crosslinked hyaluronic acid.

FIGS. 1F, G and H illustrate and interventional or percutaneous minimally invasive approach for the treatment of a patient 101 with the system 200 for placement injectable implant into the pericardial sac of the heart and fabrication of a PHC. The distal end 201 of delivery system 200 is introduced using conventional cath lab methodology into the coronary vessel 111 of the heart 100. Needle 209 crosses the patient's coronary vessel 111 via small transvascular incision or a puncture 105. Through this incision, the pericardial space 110 may be easily accessed. The distal tip 201 of the catheter then may be introduced into the incision 105. The distal tip of the catheter has an opening and is in fluid communication with the pericardial space 110. The proximal end of the catheter (cannula) 203 is connected to the delivery system 200 containing the components of the implantable PHC, such as sterile crosslinked silicone gel.

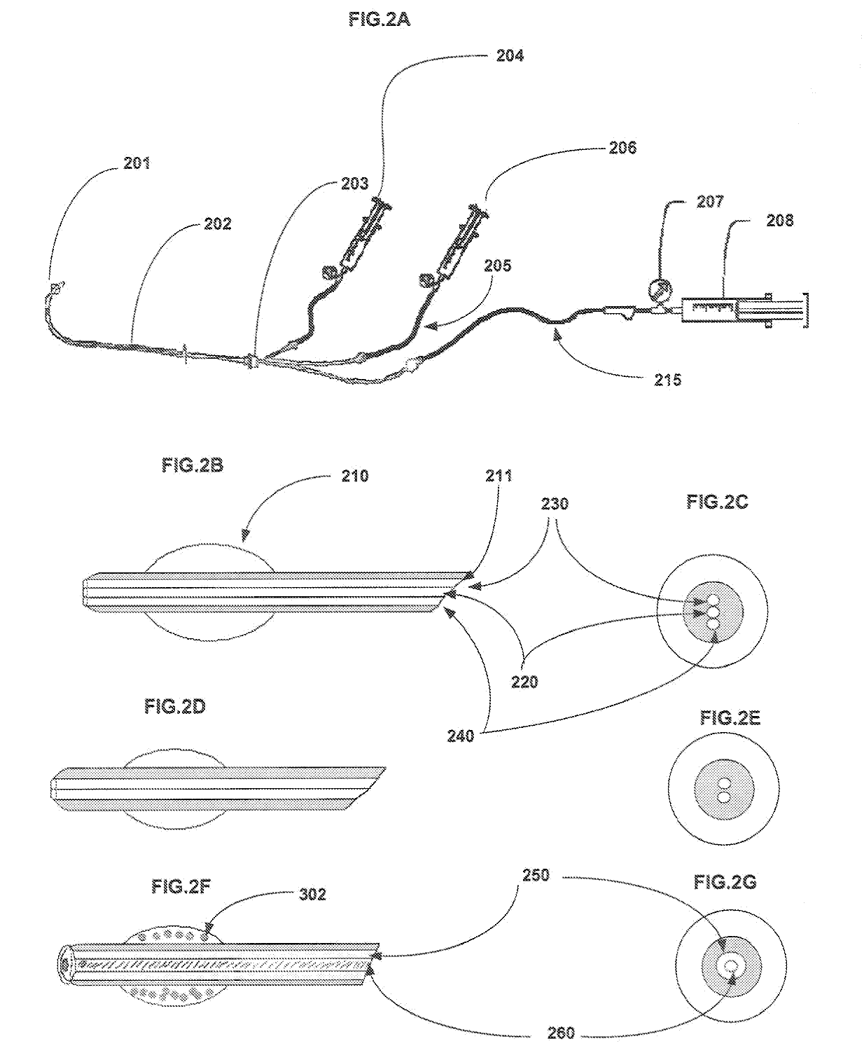

FIG. 2A is a schematic view of the cannula 202 and show the delivery system 200 components. The cannula 202 with anchoring low profile balloon 210 at the distal tip 201 communicates with sources of various components of injectable implant such as crosslinking and gelation agents, saline and the delivery apparatus 208. Delivery apparatus 207 consists of a injectable implant containing reservoir, power injector to deliver viscous substances, and pressure gauge 207 to monitor interpericardial pressure during delivery. Because some of the components for the heart constrainer formation have a viscosity range of 10000 CST to 15000 CST. The preferred volume range for injectable substance is between 40 ml and 80 ml. To conduct the delivery of such amounts of highly viscous fluids in a controlled manner a power injecting device 208 equipped with pressure gauge 207 such as Breeze inflation pump manufactured by Schneider/Namic Company can be used. Alternatively a custom made power injector can be constructed to accommodate the ergonomics of the procedure

Anchoring and sealing balloon 201 of the cannula 202 communicates with a source of saline or tissue sealant 204 via inflation lumen 211 positioned coaxially or essentially in respect with the shaft of the cannula. The inflatable anchoring balloon 210 can be inflated by infusion of saline 302 via inflation lumen 211 and utilized for the securing of the cannula. Inflation lumen is connected via two way stopcock to reservoir 204 such as a 5 ml B-D syringe containing inflation media such as saline or BioGlue. The balloon can be made out of a silicon elastomer such as Silastic and bonded using heat shrink tubing such as PTFE to the shaft of the cannula.

FIGS. 2B to 2G illustrate various configurations of the distal tip 201 of the delivery system 200 comprising inflatable anchoring and sealing balloon 210 and various lumens with specific functionality. FIGS. 2B and 2C depict a longitudinal view and a cross-sectional view respectively of a distal tip 201 of delivery system 200 with an anchoring balloon 210 and three parallel working lumens. Working lumen 230 is designed and used for the introduction of various tools for the crosslinking and gelation initiation such as fibro optics, IVAC ultrasound probe, thermo elements just to mention a few. Working lumens 220 and 240 are used for injection of the PHC components such as prepolymer and initiating agent. Working lumens 220 and 230 are communicating with the sources of PHC components: syringe 206 and injector 208. The distal ends of both lumens has an opening and are in fluid communication with the pericardial space 110.

FIGS. 2D and 2E show cross-sectional views of a distal tip 201 of delivery system 200 with an anchoring balloon 210 and just two parallel working lumens. FIGS. 2F and 2G shows a cross-sectional view of yet another possible configuration of a distal tip 201 of delivery system 200 with an anchoring balloon 210 and just two coaxial working lumens. The central lumen 260 of the is communicating with the source of PHC component 208 via injection line 215. The distal end of the central lumen 260 has an opening and is in fluid communication with the pericardial space 110. The coaxial lumen 250 of the distal tip is communicating with the source of PHC component 206 via injection line 205. The distal end of the central lumen 260 also has an opening and is in fluid communication with the pericardial space 110.

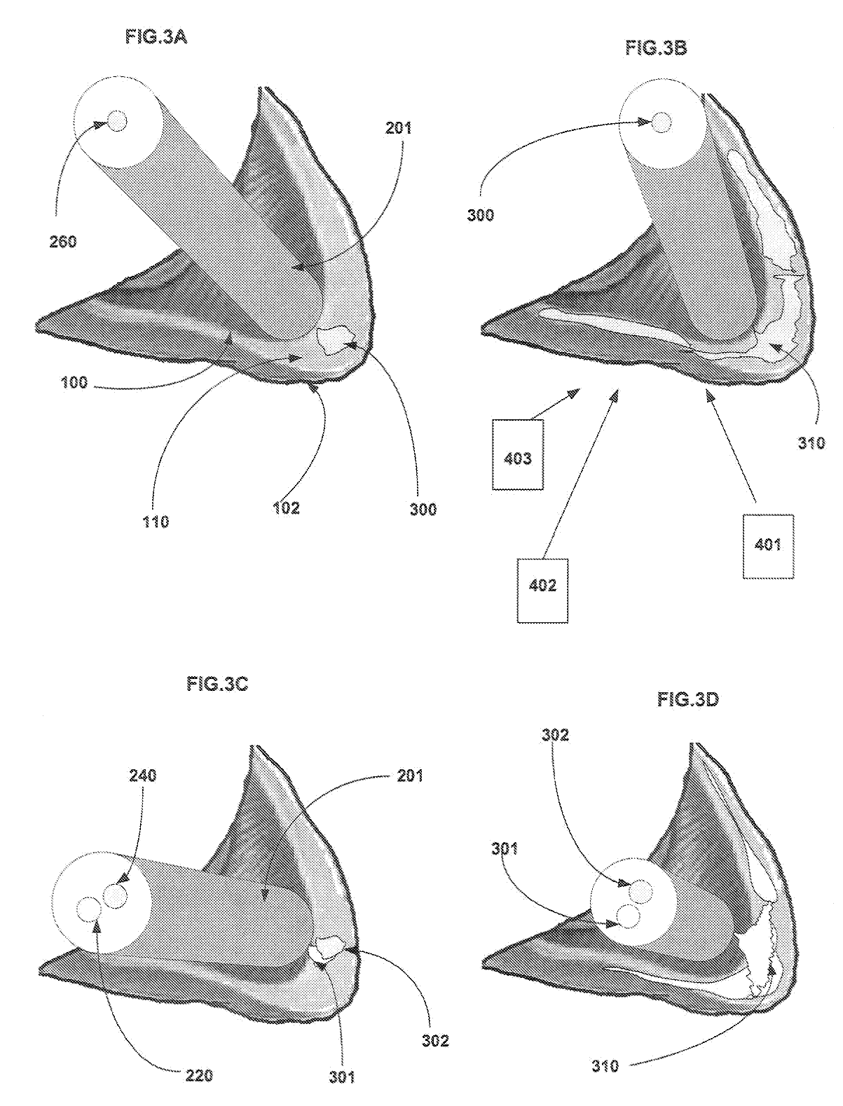

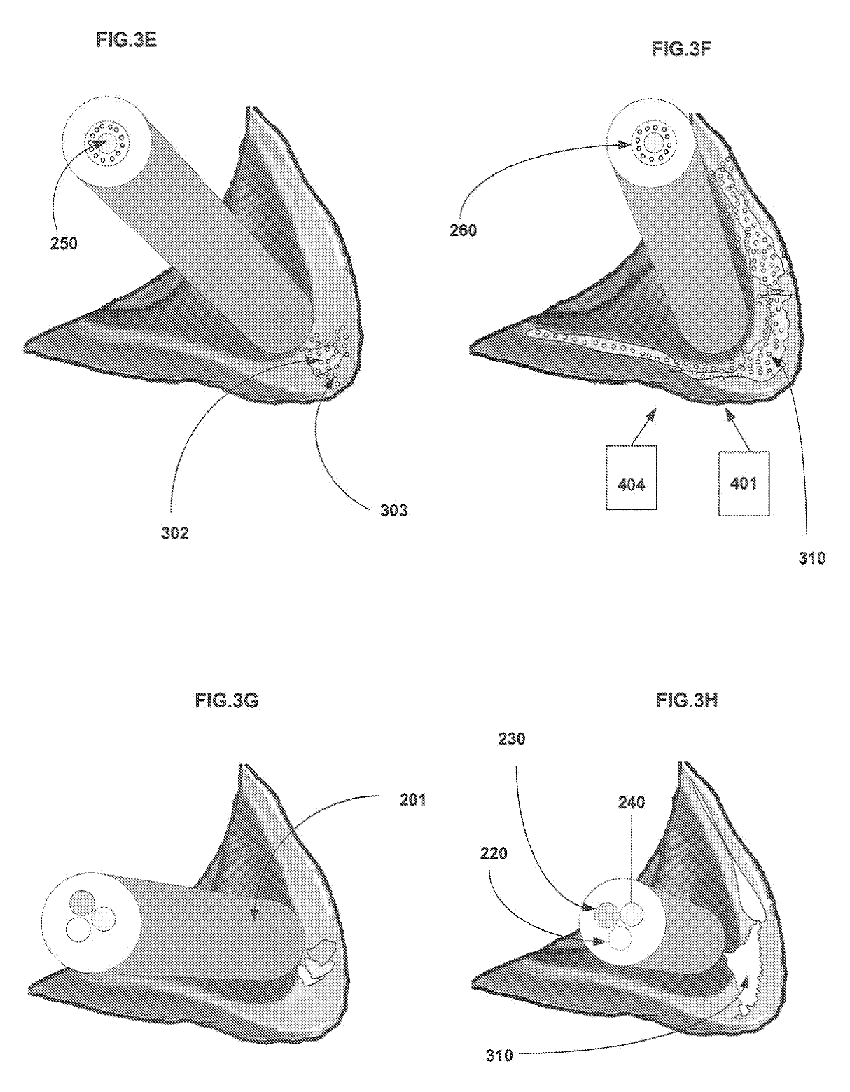

FIGS. 3A and 3B illustrate a method of fabrication of heart constrainer using just one injectable component and a single lumen at a distal tip of the catheter. The distal tip 201 is shown inserted into the pericardial space 110 of the heart. Distal tip of the cannula or the catheter secured in place by anchoring balloon 210 (not shown) resides in the space between the inner surface of the pericardium 102 and the external surface of the heart 100 defined as called pericardial space or intrapericardial space 110. Proximal end of the cannula or the catheter 203 (not shown) is connected to the delivery system outside of the patient's body. In this preferred embodiment a single working lumen 260 (shown in cross-section) is employed. The injectable substance 300 used to create a polymer network or matrix acting as a heart constrainer may be one or more biomaterials. The injectable substance 300 may include an agent or combination of agents that effects the formation of constraining network or matrix 310 under physiological conditions or upon induced conditions, typically by gelation or by cross-linking of polymeric biomaterials. The injectable substance 300 may be chosen from the variety of biopolymers and substances such as: lipids, proteins and derivatives, and polysaccharides as well as synthetic polymers. It may be natural or synthetic, biodegradable or non-biodegradable, and the polymer(s) may be further modified for enhanced properties.

The desirable injectable implant may have an array of properties allowing it to produce a therapeutic effect during a desirable therapeutic window and either to resign as a long term implant or to dissipate afterwards without any toxic product of degradation

The matrix 310 may be a hydrogel, an elastomeric crosslinked polymer, or the matrix may be made up of other materials which form a porous, fibrous network that is acting as a heart constrainer within the contemplation of this invention.

For the first preferred embodiment chosen injectable substance 300 is PLURONICS™ commercially available from BASF.PLURONICS™ or TETRONICS™, polyethylene oxide-polypropylene glycol block copolymers which are crosslinked by temperature or pH, respectively.

Suitable polymers formulations are described in greater detail in U.S. Pat. No. 5,667,778, incorporated herein by reference

Pluronics, a family of poly(ethylene oxide)-poly(propylene oxide)-poly(ethylene oxide) (PEO-PPO-PEO) triblock copolymers, are of considerable interest in the biotechnological and pharmaceutical industry for their unique surfactant abilities, low toxicity, and minimal immune response.

Aqueous solutions of Pluronic copolymers exhibit interesting temperature induced aggregation phenomena as a result of the hydrophobic nature of the PPO block. In some cases, gelation of concentrated Pluronic solutions occurs upon heating to temperatures at or just above ambient, a property which is potentially useful for medical drug delivery applications. For example, in-situ gelling materials are potentially useful as carriers for drug delivery to mucosal surfaces, i.e. the oral cavity and the respiratory, gastrointestinal, and reproductive tracts.

FIG. 3B shows final phase of the procedure when injectable substance 300 is deposited and dispersed in the pericardial space and normal physiologic conditions such as temperature 401 and pH 402 have triggered gelation process leading to the formation of the polymer matrix 310 acting as a heart constrainer. In yet another preferred embodiment, a chosen material 300 is a medical grade crosslinked silicone gel. Applied Silicone Corp., Ohio is one the manufacturers of the medical grade silicone gel.

Applied's Medical Implant Grade Responsive Silicone Gel System, PN 40004 is a two part system of pure silicone polymers designed for use in fabricating medical devices where softness, cohesiveness, and resiliency are desired. This product is supplied in two parts: base and crosslinkers. When mixed in the ratio of three parts by weight base to one part by weight crosslinkers and cured by application of heat, a soft, responsive, and cohesive gel results. The cured gel viscosity can be controlled within a limited range by varying the crosslinkers/base ratio. Heat cure cycles can be varied to tailor injectability of the gel. The mixture is then cured by application of heat. Time and temperature requirements may be tailored for the particular formulation. Partially crosslinked silicone gel may be injected into the pericardial space and immediately get receive additional crosslinking using either conventional X-ray machine or intraoperative fluoroscopy unit to prevent any migration of the gel. Irradiation can be repeated further in the form of fractionated doses post treatment for a few days under the radiation safety guidelines, to finalize the immobilization of the injected gel. FIG. 3B shows gamma radiation 403 applied to injected substance 300 to finalize formation of the polymer matrix 310 acting as a heart constrainer.

FIGS. 3C and 3D illustrate a method of fabrication of a heart constrainer using two injectable components. The distal tip 201 of an injection catheter is shown inserted into the pericardial space 110 of the heart. Two parallel working lumens 220 and 240 (shown in cross-section) are employed to convey the two injectable substance to the pericardial space. The injectable substances 301 and 302 used to create a polymer network or matrix in the pericardial space to act as a heart constrainer may be two or more agents. The chosen prepolymer is alginate. Alginate gels can develop and set at constant temperature. This unique property is particularly useful in applications involving fragile materials like cells or living tissue with low tolerance for higher temperatures. An alginate gel will develop instantaneously in the presence of divalent cations like Ca2+, Ba2+ or Sr2+ and acid gels may also develop at low pH. An alginate solution can be solidified by internal gelation method/internal setting, i.e. in situ gelling. Here a calcium salt with limited solubility, or complexed Ca2+-ions are mixed with an alginate solution into which the calcium ions are released, usually by the generation of acidic pH with a slowly acting acid such as D-glucono-α-lactone (GDL). In the preferred embodiment a chosen material 301 is a mixture of Ca3(PO4)2 and sodium alginate solution (PRONOVA™ by FMC Biopolymers and chosen material 302 is D-glucono-α-lactone (GDL).

FIGS. 3E and 3F illustrate a method of fabrication of heart constrainer using two injectable components. The distal tip 201 is inserted into the pericardial space 110 of the heart. Two coaxial working lumens 260 and 250 (shown in cross-section) are employed to inject substances 303 and 302 that when mixed together form a polymer network or matrix acting as a heart constrainer. The prepolymer in the preferred embodiment is alginate. To induce rapid gelation of the alginate triggered release of Ca 2++from liposomal compartments may be employed. In this embodiment thermally triggerable liposomes may be created by entrapping CaCl2 within liposomes constructed of 90% dipalmitoylphosphatidylcholine and 10% dimyristoylphosphatidylcholine. These liposomes released greater than 90% of entrapped Ca 2++when heated to 37.3 C. An injectable implant 300 in the form of prepolymer 302 (sodium alginate solution (PRONOVAtm by FMC Biopolymers) is injected into the patients pericardial space 110 using working lumen 250 simultaneously with a gelling agent 303 (liposome entrapped Ca2++) using working lumen 260 at room temperature but gelled rapidly when heated to 37.3 C, as a result of Ca 2++release and formation of crosslinked Ca-alginate. Patient temperature 401 elevation can be achieved by injection of the clinically used pyrogenic agents or controlled warming of the chest area. Alternatively, ultrasound 404 can be employed to triggered Ca 2++release from liposomes and therefore initiate gelation of the prepolymer such as aqueous sodium alginate in situ. Ultrasound 404 can be applied externally adjacent to the chest wall or internally via trachea or third working lumen 230.

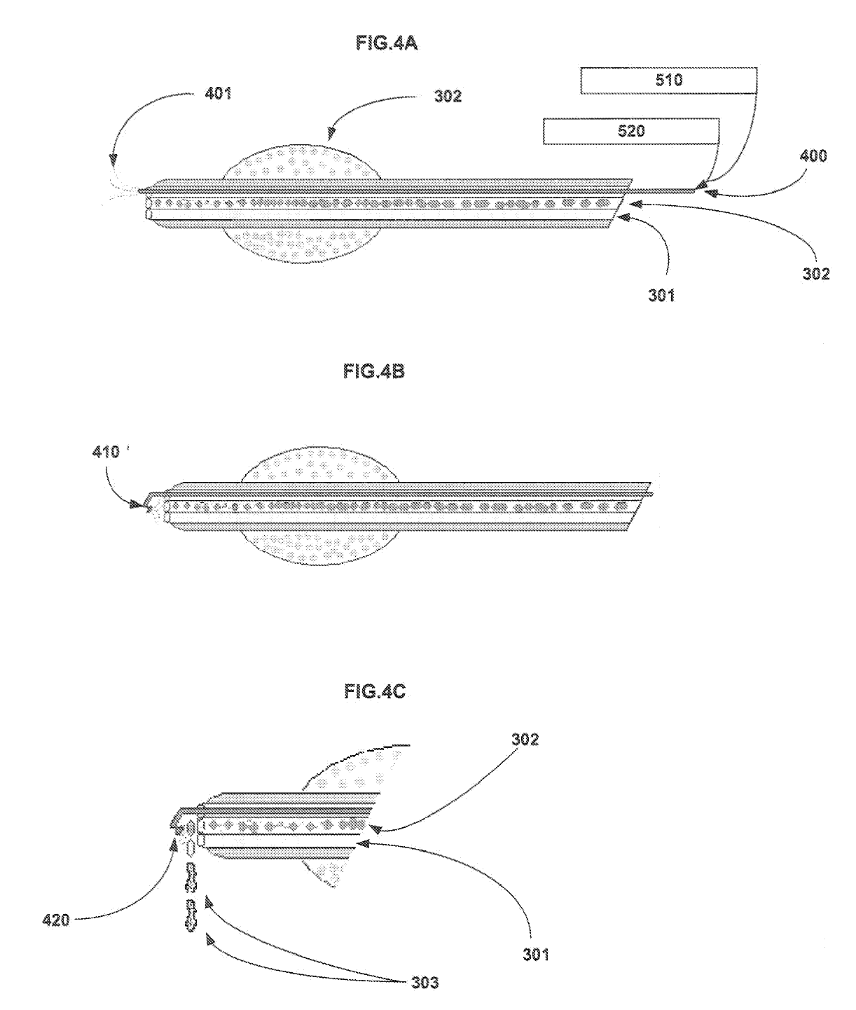

FIGS. 4A to 4C illustrate a method of fabrication of a heart constrainer using multiple lumen for the introduction of various tools for the crosslinking and gelation initiation. These lumen may be used for injection of prepolymers, a fiber optic, IVAC ultrasound probe and thermo elements.

A fiber optic conduit 400 communicating with a light source is employed for a in situ photo polymerization. The details of the distal tip 201 is shown in cross-sectional view. Three parallel working lumens 301, 302 and 400 extend the length of the catheter. lumens 301 and 302 convey the prepolymer components to the distal end of the catheter tip. The injectable substances exit lumens 301 and 302, mix and create a polymer network or matrix acting as a heart constrainer may be two or more agents. One embodiment includes the use of the photoinitiator, Quanticare QTX as a substance 302 that may initiate interfacial photopolymerization of a polyoxyethylene glycol (PEG)-co-poly(alpha-hydroxy acid) copolymer based on PEG macromonomer used as substance 301. Visible light transmitted via fiber optic 401 shines from the distal tip of a catheter into the pericardial space. The light is produced by light source 510 or laser 520. A flexible sleeve 410 is employed bend the fiber optic so as to facilitate sufficient light delivery at the openings of the working lumens 301, 302. Interaction of the prepolymer component 302 and component 301 is induced by a reflector 420 (FIG. 4C) concentrating light energy 401 on the area of contact and leads to formation of a premixture 303 that after the deposition in the pericardial space and homogenization by the heart pumping activity forms a polymer matrix 311 acting as heart constrainer.

Materials and Methods for the Matrix Formation

Various biomaterials capable of forming polymer networks and matrices, as well as physical and chemical methods of inducing intramolecular interactions leading to the such networks and matrices formation, are known to those skilled in the art and can be used in fabrication of the injectable heart constrainer. Specifically, but not limited to, hydrophilic gels (hydrogels) and hydrophobic gels can be suitable substances, for the in situ matrix formation.

Hydrogels have been shown to be instrumental for numerous medical applications ranging from crosslinked HEMA (hydroxyethyl methacrylate) used in manufacturing of the soft contact lens to calcium alginate used for cell encapsulation and wound dressings. Most recently hydrogels have become especially useful in the new field of ‘tissue engineering’ as scaffolds or matrices for repairing and regenerating a wide variety of tissues and organs. From the structural point of view hydrogels are hydrophilic polymer networks which may absorb significant amount of water and dramatically increase their volume. Hydrogels can be biodegradable and nonbiodegradble. Examples of non-biodegradable polymers include ethylene vinyl acetate, poly(meth)acrylic acid, polyamides, copolymers and mixtures thereof. Examples of biodegradable or bioerodible hydrogels described by H. S. Sawhney, C. P. Pathak and J. A. Hubell in Macromolecules, (1993) 26:581-587, the teachings of which are incorporated herein, polyhyaluronic acids, casein, collagen, gelatin, gluten, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Hydrogels can form extensive tri-dimensional networks via a number of mechanisms such as physical crosslinking and chemical crosslinking. Hydrogels are ‘reversible’, or ‘physical’ gels when the networks are held together by molecular entanglements, and/or secondary forces including ionic, H-bonding or hydrophobic forces.

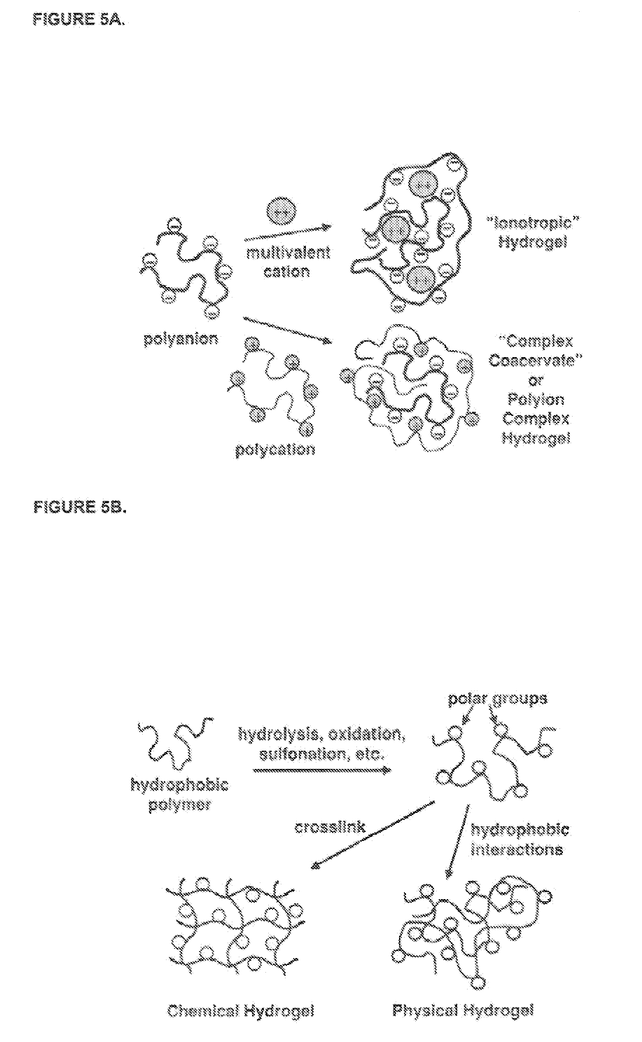

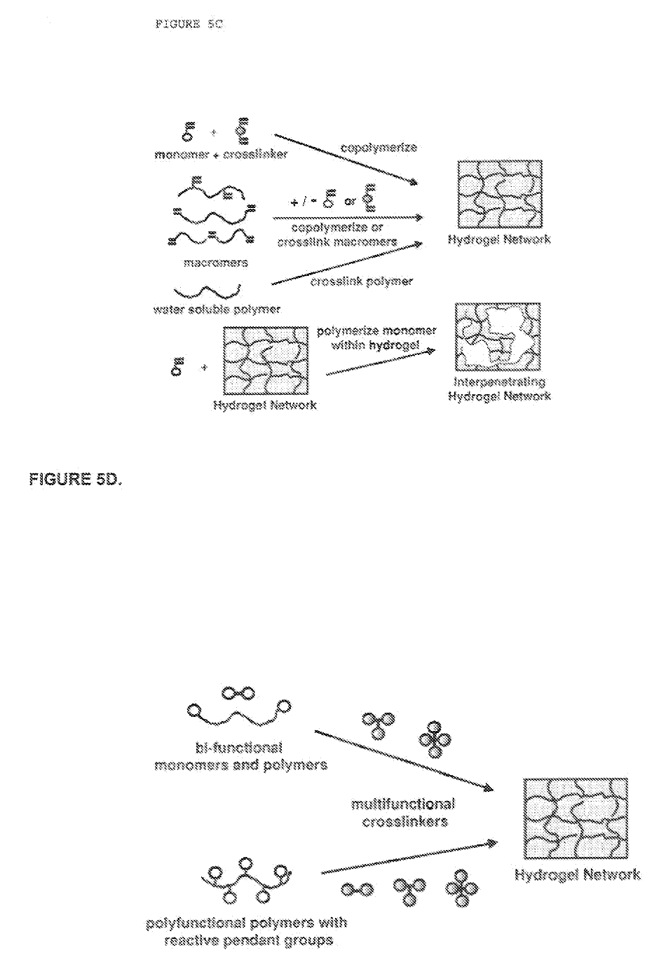

FIG. 5A is a schematic diagram of methods for formation of two types of ionic hydrogels. An example of an ‘ionotropic’ hydrogel is calcium alginate, and an example of a polyionic hydrogel is a complex of alginic acid and polylysine. For example when a polyelectrolyte is combined with a multivalent ion of the opposite charge, it may form a physical hydrogel known as an ‘ionotropic’ hydrogel. Calcium alginate is an example of this type hydrogel. Further, when polyelectrolyte of opposite charges is mixed, they may gel or precipitate depending on their concentrations, the ionic strength, and pH of the solution. The products of such ion crosslinked systems are known as complex coacervates, polyion complexes, or polyelectrolyte complexes. For example, the calcium alginate capsules developed for cell and tissue encapsulation.

(U.S. Pat. No. 4,806,355) were coated with a complex of alginate-poly (L-lysine) (PLL) in order to stabilize the capsule. Complex coacervates and polyion complex hydrogels have become especially attractive as tissue engineering matrices and scaffolds.

FIG. 5 B is a schematic of methods for formation of hydrogels by chemical modification of hydrophobic polymers. Examples of these types of hydrogels include (a) the partial hydrolysis of the acetate groups to —OH groups in conversion of PVAc to PVA, and (b) the partial hydrolysis of PAN to a polymer containing varying concentrations of acrylonitrile, amide and carboxyl pendant groups. In either case the resulting gel may be subsequently covalently crosslinked Hydrogels are ‘permanent’ or ‘chemical’ gels when they are covalently-crosslinked networks. The first synthetic hydrogels designed by Wichterle and Lim [Hydrophilic gels in biologic use, Nature 185 (1960) 117.] were based on copolymerization of HEMA (hydroxyethyl methacrylate) with crosslinker EGDMA (ethylene glycol dimethacrylate). Chemical hydrogels may also be generated by crosslinking of water-soluble polymers, or by conversion of hydrophobic polymers to hydrophilic polymers plus crosslinking to form a network.

FIG. 5C is a schematic diagram of methods for formation of crosslinked hydrogels by free radical reactions, including a variety of polymerizations and crosslinking of water-soluble polymers. Examples include crosslinked PHEMA and PEG hydrogels.

FIG. 5D is a schematic diagram of methods for formation of crosslinked hydrogels by condensation reactions of multifunctional reactants. Examples of the reactant groups include reactions of (a) isocyanates and amines or alcohols to form urea or urethane bonds, (b) amines or thiols and vinyl groups to form amines or sulfides by Michael additions, (c) amines and active esters such as N-hydroxy succinimide to form amides, (d) acids or acid chlorides and alcohols to form esters, (e) aldehydes and amines to form Schiff bases, etc. Typical examples of natural and synthetic polymers that are used to form hydrogels by such condensation reactions include many different types of polysaccharides, collagen, PAAc, PVA and PEG.

There are many different macromolecular structures that are possible for physical and chemical hydrogels. They include the following: crosslinked or entangled networks or linear homopolymers; linear copolymers, and block or graft copolymers; polyion-multivalent ion, polyion-polyion or H-bonded complexes, hydrophilic networks stabilized by hydrophobic domains; and IPNs (interpenetrating network) or physical blends. Hydrogels may also have various physical forms, including solid matrices (e.g., soft contact lenses), microparticles (e.g., microbeads for wound exudates), injectable gels (e.g., tissue anti adhesives, bio glues), and liquids (e.g., that form gels under certain conditions, i.e. heating, radiation, etc.). A wide array of polymer compositions have been used to fabricate hydrogels. The compositions can be divide into natural polymer hydrogels, synthetic polymer hydrogels and combination of the two categories.

For the simplicity, Table 1 presents industrial abbreviation are used instead of full chemical nomenclature names.

TABLE 1
Abbreviations

| | |
|---|---|
| CD for cyclodextrin | DX for p-dioxanone |
| EG for ethylene glycol | EGDMA for ethylene glycol |
| | dimethacrylate |
| HA, for hyaluronic acid | HEMA for hydroxyethyl |
| | methacrylate |
| IPN for interpenetrating network | MBAAm for |
| | methylene-bis-acrylamide |
| PAAc for poly(acrylic acid) | PAAm for polyacrylamide |
| PAN for polyacrylonitrile | PBO for poly(butylene oxide) |
| PCL for polycaprolactone | PEG for poly(ethylene glycol) |
| PEI for poly(ethylene imine) | PEO for poly(ethylene oxide) |
| PEMA for poly(ethyl methacrylate) | PF for propylene fumarate |
| PGEMA for poly(glucosylethyl | PHB for poly(hydroxy butyrate) |
| methacrylate) | |
| PHEMA for poly(hydroxyethyl | PHPMA for poly(hydroxypropyl |
| methacrylate) | methacrylamide) |
| PLA for poly(lactic acid) | PLGA for |
| | poly(lactic-co-glycolic acid) |
| PMMA for poly(methyl methacrylate) | PNIPAAm for poly(N-isopropyl |
| | acrylamide) |
| PNVP for poly(N-vinyl pyrrolidone) | PPO for poly(propylene oxide) |
| PVA for poly(vinyl alcohol) | PVAc for poly(vinyl acetate) |
| PVamine for poly(vinyl amine) | |

Polymers commonly used in medical applications are represented include:

(I) Natural polymers and their derivatives: Anionic polymers: HA, alginic acid, pectin, carrageenan, chondroitin sulfate, dextran sulfateCationic polymers: chitosan, polylysineAmphipathic polymers: collagen (and gelatin), carboxymethyl chitin, fibrinNeutral polymers: dextran, agarose, pullulan.

(II) Synthetic polymers:Polyesters: PEG-PLA-PEG, PEG-PLGA-PEG, PEG-PCL-PEG, PLA-PEG-PLA, PHB, P(PEG/PBO terephthalate)

(III) Other polymers:PEG-bis-(PLA-acrylate), PEG-g-P(AAm-co-Vamine), PAAm, P(NIPAAm-co-Aac), P(NIPAAm-co-EMA), PVAc/PVA, PNVP, P(MMA-co-HEMA), P(AN-co-allyl sulfonate), P(biscarboxy-phenoxy-phosphazene), P(GEMA-sulfate)

(IV) Combinations of natural and synthetic polymers:P(PEG-co-peptides), alginate-g-(PEO-PPO-PEO), P(PLGA-co-serine), collagen-acrylate, alginate-acrylate, P(HPMA-g-peptide), P(HEMA/Matrigel™), HA-g-NIPAAm

A variety of prepolymers, precursors, and principal components for hydrogel fabrication are commercially available. Just to list a few: Purified natural and some chemically modified Cyclodextrins are available in from BioResearch Corporation of Yokohama (BICO), Japan. In the US the biggest manufacturer of the cyclodextrin is Cyclodextrin Technologies Development, Inc. (CTD) offering variety of cyclodextrin products under the trade name Trappsol™. One of the largest US producer of hyaluronic acid Genzyme, offers HyluMed™ product line comprised of sterile and medical-grade HA powder available in a broad range of molecular weights to meet the diverse industrial needs.

Du Pont is one the world wide largest manufacturers of polyvinyl alcohol, under the trade name Elvanol, polyvinyl acetate, under the trade name Elvacet, polymethyl methacrylate and polyethyl methacrylate, under the trade name Elvacite. Nova Matrix, division of FMC Biopolymer, Norway is the worlds leading producer and supplier of ultrapure sodium alginate under the trade name PRONOVA™ and water-soluble chitosan salts under the trade name PROTOSAN™.

Various methods for hydrogels fabrication are known to those skilled in the art and are used in industry and science. Some of them are shown schematically in Diagrams 3A-3D and listed in Tables 3 and 4. Hydrogels are described in more detail in Hoffman, D. S., “Polymers in Medicine and Surgery,” Plenum Press, New York, pp 33-44 (1974).

Methods utilized for the formation of the “physical gels” include: Warm a polymer solution to form a gel (e.g., PEO-PPO-PEO block copolymers in H2O); Cool a polymer solution to form a gel (e.g., agarose or gelatin in H2O) Lower pH to form an H-bonded gel between two different polymers in the same aqueous solution (e.g., PEO and PAAc); Mix solutions of a polyanion and a polycation to form a complex coacervate gel (e.g., sodium alginate plus polylysine); and Gel a polyelectrolyte solution with a multivalent ion of opposite charge (e.g., Na+ alginate-+Ca2++2Cl—).

Methods utilized for the formation of the “chemical gels” include: Crosslink polymers in the solid state or in solution with:Radiation (e.g., irradiate PEO in H2O) Chemical crosslinkers (e.g., treat collagen with glutaraldehyde or a bis-epoxide) Multi-functional reactive compounds (e.g., PEG+diisocyanate=PU hydrogel); Copolymerize a monomer+plus crosslinker in solution (e.g., HEMA+EGDMA); Copolymerize a monomer+a multifunctional macromer (e.g., bis-methacrylate terminated PLA-PEO-PLA+photosensitizer+visible light radiation); Polymerize a monomer within a different solid polymer to form an IPN gel (e.g., AN+starch); and Chemically convert a hydrophobic polymer to a hydrogel (e.g., partially hydrolyse PVAc to PVA or PAN to PAN/PAAm/PAAc)

An example of a hydrophobic gels is silicone gel. Silicones, or “polysiloxanes”, are inorganic polymers consisting of a silicon-oxygen backbone(—Si—O—Si—O—Si—O—) with side groups attached to the silicon atoms. Certain organic side groups can be used to link two or more of these —Si—O— backbones together. By varying the —Si—O— chain lengths, side groups, and crosslinking, silicones can be synthesized into a wide variety of materials. They can vary in consistency from liquid to gel to rubber. The most common type is linear polydimethylsiloxane or PDMS. Silicones have been widely used as inert and non toxic biomaterial for various medical applications, including silicone gel-filled breast prosthesis. A single-lumen silicone gel-filled breast prosthesis is a silicone rubber shell made of polysiloxane(s), such as polydimethylsiloxane and polydiphenylsiloxane. The shell either contains a fixed amount cross-linked polymerized silicone gel, filler, and stabilizers or is filled to the desired size with injectable silicone gel at time of implantation. The device is intended to be implanted to augment or reconstruct the female breast The silicone gel contained in gel-filled silicone breast implants, including the microscopic “bleed” of silicone particles across the implant membrane, is not associated with the complications caused by the free injection of liquid silicone into the breast.

Medical-grade Liquid Injectable Silicone has been used in a variety of medical applications for many decades. LIS is a clear, colorless, highly purified, thick liquid. Because it is sterile and non-allergenic, normally no test injection is required. The therapeutic value of microdroplet LIS for building soft-tissue is well established. For over 40 years, it has received wide support in the medical literature. In December 1998, a specially appointed National Science Panel (composed of four eminent scientists from the disciplines of immunology, epidemiology, toxicology and rheumatology reported its unanimous conclusion that there was no evidence linking silicone in breast implants to any systemic disease. The Panel's report was based on a yearlong analysis of the most rigorously tested and relevant scientific information available.

Silicone gels have lightly cross-linked polysiloxane networks, swollen with PDMS fluid to produce a cohesive mass. The PDMS fluid is not chemically bound to the crosslinked network but is retained only by physical means, as water is in a sponge, and there is a tendency for the fluid to “bleed”. The degree of cross-linking and amount of fluid affects the physical properties of the gel and the rate at which fluid “bleeds” from it. Once suitably cross-linked, silicone gels retain their form without external containment.

The degree of cross-linking of the gel can be increased using chemical or physical methods, therefore stabilizing and preventing any migration in the body gel based implants. Two principal physical methods of degree of crosslinking increase are temperature and radiation. Medical grade silicone gel are commercially available by a number of supplies. Just to mention a few: NuSil Silicone Technology, CA and Applied Slicone Corp., OH.

Injectable Microparticles

An injectable implant comprising microparticles in solution (a dispersion) may used as a heart constrainer. The microparticles may be a predetermined range of about 1 to about 200 microns. In one embodiment, the microparticles may be 20 microns or less. In a preferred embodiment, the microparticles may be 10 microns or less. The microparticle size delivered to the patients pericardial space may be determined by the delivery method used. One suspending solution for the microparticles may be water. On the other hand, the suspending solution may also be a solvent, for example dimethylsulfoxide (DMSO) or ethanol adjuvants.

In one embodiment, a suspending solution along with the microparticles may be introduced to as a dispersion to the patients pericardial space and the microparticles remain in the space as the solution dissipates into the surrounding tissue. Thus, the microparticles deposited and distributed in the entire pericardial sac will act as a heart constrainer. In one embodiment, the dispersion (detailed above) may be injected in to the pericardial space during via a minimally invasive procedure, subxiphoidal or percutaneous. Besides just a mechanical constraining of the heart, microparticle based implant can also facilitate a sustained or controlled drug release. One embodiment of a composition suitable for the described method includes the use of a bioerodible microparticles coupled with a therapeutic or biologically active agent. The bioerodible microparticle may consist of a bioerodible polymer such as poly (lactide-co-glycolide). The composition of the bioerodible polymer is controlled to release a therapeutic or biologically active agents over a period of 1-2 weeks. In one preferred embodiment of a composition, the bioerodible microparticle may be a PLGA polymer 50:50 with carboxylic acid end groups. PLGA is a base polymer often used for controlled release of drugs and medical implant materials (i.e. anti-cancer drugs such as anti-prostate cancer agents). Two common delivery forms for controlled release include a microcapsule and a microparticle (e.g. a microsphere). The polymer and the agent are combined and usually heated to form the microparticle prior to delivery to the site of interest (Mitsui Chemicals, Inc). In one embodiment, the PLGA polymer 50:50 with carboxylic acid end groups harbors an anti arrhythmic drug for slow release. It is preferred that each microparticle may release at least 20 percent of its contents and more preferably around 90 percent of its contents. In one embodiment, the microparticle harboring at least one angiogenic and/or therapeutic agent will degrade slowly over time releasing the agent or release the agent immediately upon placement into the patients pericardial space in order to rapidly effect the patient. In another embodiment, the microparticles may be a combination of controlled-release microparticles and immediate release microparticles. A preferred rate of deposition of the delivered factor will vary depending on the condition of the subject undergoing treatment.

Another embodiment of a composition suitable for the described method includes the use of non-bioerodible microparticles that may harbor one or more of the biologically active agents. The agents may be released from the microparticle by controlled-release or rapid release. The microparticles may be placed directly in the pericardial space. The non-bioerodible microparticle may consist of a non-bioerodible polymer such as an acrylic based microsphere for example a tris acryl microsphere (provided by Biosphere Medical). In another embodiment, non-bioerodible microparticles may be used alone or in combination with another polymer or matrix forming component. In addition, non-bioerodible microparticles may be used to reinforce hydrogel based matrix acting as heart constrainer. In one embodiment, non-bioerodible microparticles may be used alone or in combination with an agent to treat pain and/or arrhythmias.

Methods of Delivery and Administration

Cannula or catheter may be used to deliver the any one or multiple components of the embodiments to the pericardial space. Several catheters have been designed in order to precisely deliver agents to a major areas within the heart. Several of these catheters have been described (U.S. Pat. Nos. 6,309,370; 6,432,119;). The delivery device may include an apparatus for intracardiac drug administration. The apparatus may include, for example, a catheter body capable of traversing a blood vessel and a dilatable balloon assembly coupled to the catheter body comprising a balloon having a proximal wall. A needle may be disposed within the catheter body and includes a lumen having dimensions suitable for a needle to be advanced there through. The needle body includes an end coupled to the proximal wall of the balloon. The apparatus may be suitable for accurately introducing a injectable implants in the form of prepolymer or a few prepolymer plus network formation inducing agent(s) into the patient's pericardial space. “Prepolymer” means a macromer or polymer composition that forms a hydrogel upon exposure to some initiation event, such as crosslinking or gelation. In order to accommodate pericardial placement of the injectable implant comprising more then one component, multilumen delivery system may be utilized, where components (prepolymers) are contained separately prior to initiation of injection. The injectable implant can be formed in any manner, generally from a prepolymer, that is brought into contact with an initiator within the device or at the very exit out of the device. The prepolymer component(s) are fed to the device from syringes or other containment reservoirs and the implant composition can be formed by combining at the distal tip of the delivery catheter or cannula and immediate ejection from the front end (distal end) of the delivery system. The implant composition can also be formed without combining the components at the distal tip by simultaneous ejection of the components from the distal tip of the delivery system into the pericardial space. In one embodiment, the implant is formed by bringing together two liquid components that form a hydrogel upon extruding or pushing both components out of the device. In another embodiment, the implant can be formed by having an initiator within the device, wherein the prepolymer contacts the initiator, the hydrogel forms at exit of the distal tip, and is then ejected from the device. In any case the invention is not utilizing any particular premixing methods or mechanisms since actual mixing and homogenization takes place in the pericardial sac due to the pumping function of the heart that acts as a natural homogenizer.

In a preferred embodiment, the method involves bringing together two liquid components within a dual lumen catheter, having a dual tip on the end. A variety of configurations of the two liquid components is possible. In one embodiment, the two liquid components may each contain prepolymer, whereupon the prepolymers form the hydrogel when mixed. In another embodiment, the two liquid components may each contain prepolymers and one or both components may contain a crosslinking initiator. In another embodiment, the prepolymer may be contained in only one component, while one or both components contain a crosslinking initiator. Or, the prepolymer may be in one component, while the initiator is in the other component. In any event, a hydrogel is formed when the respective components are brought in contact. The hydrogel formation from one or more prepolymers and macromers are described in WO 01/68720 to BioCure, Inc. and U.S. Pat. No. 5,410,016 to Hubbell et al. The hydrogel string formation from one or more prepolymers and macromers using a string forming and extruding device with premixing chamber is described in U.S. Patent Application No 20040247867 to Hassan et al.

In one embodiment, the apparatus includes a first annular member having a first lumen disposed about a length of the first annular member, and a second annular member coupled to the first annular member having a second lumen disposed about a length of the second annular member, wherein collectively the first annular member and the second annular member have a diameter suitable for placement at a treatment site within a mammalian body. Representatively, distal ends of the first annular member and the second annular member are positioned with respect to one another to allow a combining of treatment agents introduced through each of the first annular member and the second annular member to allow a combining of treatment agents at the treatment site. Such an apparatus is particularly suitable for delivering a multi-component matrix forming material e.g., individual components through respective annular members that forms a polymer network acting as a heart constrainer into entire pericardial space. In the embodiments described herein, a substance delivery device and a method for delivering a substance are disclosed. The delivery device and method described are suitable, but not limited to, local drug delivery in which a treatment agent composition (possibly including multiple treatment agents and/or a sustained-release composition) is introduced into the injectable heart implant prior to or at the time of the injection into pericardium. The preferred period for sustained release of one or more agents is for a period of one to twelve weeks, preferably two to eight week. Suitable therapies include, but are not limited to, delivery of drugs for the treatment of pain, arrhythmia, as well as agents for the therapeutic induction of angiogenesis. In another embodiment, a method may include introducing a Radiopaque or Echogenic agent in a injectable composition, to provide a better visualization of the injectable implant distribution in the pericardial sac.

An array of guiding modalities may be used to facilitate an accurate insertion of the delivery system front end such as cannula or a tip of the catheter into the pericardial space. An imaging modality may be used such as a contrast-assisted fluorescent scope that permits a cardiologist to observe the placement of the catheter tip or other instrument within the pericardial space. The contrast-assisted fluoroscopy utilizes a contrast agent that may be injected into heart chamber and then the area viewed under examination by a scope, thus the topography of the injection site is more easily observed and may be more easily treated (U.S. Pat. Nos. 6,385,476 and 6,368,285). Suitable imaging techniques include, but are not limited to, ultrasonic imaging, optical imaging, and magnetic resonance imaging for example Echo, ECG, SPECT, MRI, and Angiogram.

Induction of Crosslinking, Gelation and Other Polymer Network Forming Mechanisms

Gelation of the prepolymer can be via a number of mechanisms, such as physical crosslinking or chemical crosslinking. Physical crosslinking includes, but is not limited to, complexation, hydrogen bonding, desolvation, Van der Waals interactions, and ionic bonding. Chemical crosslinking can be accomplished by a number of means including, but not limited to, chain reaction (addition) polymerization, step reaction (condensation) polymerization and other methods of increasing the molecular weight of polymers/oligomers to very high molecular weights. Other methods of increasing molecular weight of polymers/oligomers include but are not limited to polyelectrolyte formation, grafting, ionic crosslinking, etc. Various crosslinkable groups are known to those skilled in the art and can be used, according to what type of crosslinking is desired. For example, hydrogels can be formed by the ionic interaction of divalent cationic metal ions (such as Ca.sup.+2 and Mg.sup.+2) with ionic polysaccharides such as alginates, xanthan gums, natural gum, agar, agarose, pectin, and amylopectin. Multifunctional cationic polymers, such as poly(1-lysine), poly(allylamine), poly(ethyleneimine), poly(guanidine), poly(vinyl amine), which contain a plurality of amine functionalities along the backbone, may be used to further induce ionic crosslinks. Hydrophobic interactions are often able to induce physical entanglement, especially in polymers, that induces increases in viscosity, precipitation, or gelation of polymeric solutions. Block and graft copolymers of water soluble and insoluble polymers exhibit such effects, for example, poly(oxyethylene)-poly(oxypropylene) block copolymers, copolymers of poly(oxyethylene) with poly(styrene), poly(caprolactone), poly(butadiene), etc.

Other means for gelation also may be advantageously used with prepolymers that contain groups that demonstrate activity towards functional groups such as amines, imines, thiols, carboxyls, isocyanates, urethanes, amides, thiocyanates, hydroxyls, etc. Desirable crosslinkable groups include (meth)acrylamide, (meth)acrylate, styryl, vinyl ester, vinyl ketone, vinyl ethers, etc. Particularly desirable are ethylenically unsaturated functional groups.

The hydrogel can be formed from one or more macromers (crosslinkable macromonomer) that include a hydrophilic or water soluble region and one or more crosslinkable regions. The macromers may also include other elements such as one or more degradable or biodegradable regions. A variety of factors-primarily the desired characteristics of the formed hydrogel—determines the most appropriate macromers to use. Many macromer systems that form biocompatible hydrogels can be used.

Macromers can be constructed from a number of hydrophilic polymers, such as, but not limited to, polyvinyl alcohols (PVA), polyethylene glycols (PEG), polyvinyl pyrrolidone (PVP), polyalkyl hydroxy acrylates and methacrylates (e.g. hydroxyethyl methacrylate (HEMA), hydroxybutyl methacrylate (HBMA), and dimethylaminoethyl methacrylate (DMEMA)), polysaccharides (e.g. cellulose, dextran), polyacrylic acid, polyamino acids (e.g. polylysine, polyethylmine, PAMAM dendrimers), polyacrylamides (e.g. polydimethylacrylamid-co-HEMA, polydimethylacrylamid-co-HBMA, polydimethylacrylamid-co-DMEMA). The macromers can be linear or can have a branched, hyperbranched, or dendritic structure.

Methods of the Matrix Formation Based on External Energy Application

I. Thermally Triggered Matrix Formation

Many thermal reversible materials may be used for in situ fabrication of the heart constrainer. Generally, thermal reversible components at temperatures of approximately 37 degrees Celsius and below are liquid or soft gel. When the temperature shifts to 37 degrees Celcius or above, the thermal reversible components tend to harden. In one embodiment, the temperature sensitive matrix forming component may be triblock poly (lactide-co-glycolide)-polyethylene glycol copolymer. This is commercially available (REGEL™ Macromed, Utah). In another embodiment, the temperature sensitive matrix forming component may include the following consisting of poly (N-isopropylacrylamide) and copolymers of polyacrylic acid and poly (N-isopropylacrylamide). Another temperature sensitive matrix forming component commercially available is PLURONICS™ (aqueous solutions of PEO-PPO-PEO (poly(ethylene oxide)-poly(propylene oxide)-poly(ethylene oxide) tri-block copolymers BASF, N.J.) (Huang, K. et al. “Synthesis and Characterization of Self-Assembling Block copolymers Containing Bioadhesive End Groups” Biomacromolecules 2002,3,397-406). Patient temperature elevation can be achieved by injection of the clinically used pyrogenic agents or controlled warming of the chest area in order to activate in situ gel matrix formation.

II. Thermally and Ultrasonically Triggered Encapsulated Activator Release

Other means for gelation and crosslinking were the initiator encapsulated in micro containers such as microbubbles or liposomes may be utilized as a part of muti-component system activated by various physical mechanisms such as temperature and ultrasound. Triggered release of calcium from lipid vesicles for rapid gelation of polysaccharide and protein hydrogels was described by Eric Westhaus 1, Phillip B. Messersmith (Biomaterials 22 (2001) 453}462)

Triggered release of Ca 2++from liposomal compartments may be employed to induce rapid gelation of polysaccharide and protein-based hydrogels. For example thermally triggerable liposomes may be created by entrapping CaCl2 within liposomes constructed of 90% dipalmitoylphosphatidylcholine and 10% dimyristoylphosphatidylcholine. These liposomes released greater than 90% of entrapped Ca 2++when heated to 37.3 C. An injectable implant in the form of precursor agent containing liposomes suspended in a prepolymer (aqueous sodium alginate) may be injected into the patients pericardial space at room temperature but gelled rapidly when heated to 37.3 C, as a result of Ca 2++release and formation of crosslinked Ca-alginate. Patient temperature elevation can be achieved by injection of the clinically used pyrogenic agents or controlled warming of the chest area.

Alternatively, ultrasound can be employed to triggered Ca 2++release from liposomes and therefore initiate gelation of the prepolymer such as aqueous sodium alginate in situ. Ultrasound can be applied externally across the chest wall or internally via trachea.

III. Gamma Radiation and X-Rays for the In Situ Polymer Network Formation.

Convenient method of radiation-based synthesis of hydrogels is the irradiation of polymers in aqueous solution, since such systems, containing neither monomers nor crosslinking agents (otherwise frequently used to enhance gel formation), are easier to control and study. Also, with the application of this method, lower number of usually unwanted processes occurs, as e.g. homografting of monomer on a polymer chain that may lead to branched structures. [Inokuti M.; Gel formation in polymers resulting from simultaneous crosslinking and scission; J. Chem. Phys., 38, 2999 (1963).]. Typical examples of simple, synthetic polymers used for hydrogel formation by this method are poly(vinyl alcohol)—PVAL, polyvinylpyrrolidone—PVP, poly(ethylene oxide)—PEO, polyacrylamide—PAAm, poly(acrylic acid)—PAA and poly(vinyl methyl ether)—PVME

A number of polymers including but not limited to collagen, gelatin and silicone can be additionally crosslinked using gamma radiation and X-rays. In one of the embodiments of this invention medical grade crosslinked silicone gel is injected into the pericardial space and immediately crosslinked using either conventional X-ray machine or intraoperative fluoroscopy unit to prevent any migration of the gel. Irradiation can be repeated further in the form of fractionated doses post treatment for a few days under the radiation safety guidelines, to finalize the immobilization of the injected gel.

IV. Light Induced Photo-Polymerization

In yet another embodiment photo-polymerizable hydrogels may be used to form pericardial heart constrainer. A number of hydrogels are used in tissue engineering applications. These gels are biocompatible and do not cause thrombosis or tissue damage. These hydrogels may be photo-polymerized in situ in the presence of ultraviolet (UV) or visible light depending on the photo initiation system. Photo-polymerizing materials may be spatially and temporally controlled by the polymerization rate. These hydrogels have very fast curing rates. A monomer or macromer form of the hydrogel may be introduced to the pericardial space with a photo initiator. Examples of these hydrogel materials include PEG acrylate derivatives, PEG methacrylate derivatives or modified polysaccharides.

Visible light maybe used to initiate interfacial photopolymerization of a polyoxyethylene glycol (PEG)-co-poly(alpha-hydroxy acid) copolymer based on PEG macromonomer in the presence of an initiator for example Quanticare QTX. Initiator 2-hydroxy-3-[3,4,dimethyl-9-oxo-9H-thioxanthen-2-yloxy]N,N,N-trimethyl-1-propanium chloride photo-initiator may be obtained as Quantacure QTX. This is a specific water-soluble photo-initiator that absorbs ultraviolet and/or visible radiation and forms an excited state that may subsequently react with electron-donating sites and may produce free radicals. This technology has been used to demonstrate adherence to porcine aortic tissue, resulting in a hydrogel barrier that conformed to the region of introduction. The resulting matrix was optimized in vitro and resulted in the formation of a 5-100 microns thick barrier (Lyman, M D et. al. “Characterization of the formation of interfacially photopolymerized thin hydrogels in contact with arterial tissue Biomaterials” 1996 February; 17 (3):359-64).

The source of the UV or visible light may be supplied by means of a catheter for example a fiber optic tip catheter or lead on a catheter. In this embodiment, the minimally invasive procedure including both subxiphoid and percutaneous approaches may be used to deliver the components for the implant fabrication and the light source to the patients pericardial space. The catheter may be designed to provide a delivery device with at least one lumen for one or more implant forming agent(s) and a light source for initiation of photo-polymerizing agent upon its extrusion from the distal tip. One embodiment includes the use of the photoinitiator, Camphorquinone that may facilitate the cross-linking of the hydrogel by a light on the tip of a catheter within the pericardial space. Another embodiment includes the use of the photoinitiator, Quanticare QTX that may facilitate the cross-linking of the hydrogel by a light on the tip of a catheter within pericardial space. Another embodiment includes the use of a catheter with a UVA light source to induce the polymerization event in the presence of a light sensitive initiator. Other initiators of polymerization in the visible group include water soluble free radical initiator 2-hydroxy-3-[3,4, dimethyl-9-oxo-9H-thioxanthen-2-yloxy]N,N,N-t-rimethyl-1-propanium chloride. This cascade of events provides the necessary environment for initiation of polymerization of suitable vinyl monomers or pre-polymers in aqueous form within the pericardial space (Kinart et. al. Electrochemical atudies of 2-hydroxy-3-(3,4-dimethyl-9-ox-o-9H-thioxanthen-2-yloxy)N,N,N-trimethyl-1-propanium chloride” J. Electroanal. Chem 294 (1990) 293-297).

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

We claim:

1. A method to constrain a heart comprising:
injecting a biopolymer into a pericardial space of the heart;
inducing intramolecular or intermolecular interactions in the biopolymer in the pericardial space to modify physical properties of the biopolymer in the pericardial space, and
constraining the heart with the modified biopolymer in the pericardial space.

2. A method as in claim 1 wherein inducing intramolecular interactions comprises at least one of polymerization, crosslinking and gelation of the biopolymer.

3. A method as in claim 1 wherein the biopolymer comprises the biopolymer forming a polymer network in the pericardial space.

4. A method as in claim 1 wherein the biopolymer comprises a crosslinked silicone gel and inducing intramolecular interactions comprises increasing a degree of crosslinking of the gel.

5. A method as in claim 4 wherein increasing a degree of crosslinking is accomplished by applying radiation to the biopolymer in the pericardial space.

6. A method as in claim 4 wherein increasing a degree of crosslinking is accomplished by applying heat to the biopolymer in the pericardial space.

7. A method as in claim 1 wherein the solidified biopolymer forms an polymer matrix around heart and the matrix constrains the heart.

8. A method as in claim 1 further comprising deploying the biopolymer throughout the entire pericardial space.

9. A method as in claim 1 further comprising forming a transpericardial incision, placing a cannula through the incision to inject the synthetic biopolymer, and sealing the incision after extraction of the cannula.

10. A method as in claim 1 further comprising injecting the synthetic biopolymer as a multiple component compound, wherein at least two of the components are injected separately and combined in the pericardial space.

11. A method for placing a heart constrainer in a patient comprising:
placement of a cannula for the injection of an implant in a pericardial space of the heart, including forming a transpericardial incision through which the cannula is inserted;
connecting to a proximal section of the cannula of an implant delivery system;
preparing components of an injectable constrainer for injection by the implant delivery system;
controlling injection of the components of the injectable constrainer by the delivery system through the cannula and into the pericardial space;
induction of the injected constrainer components to form a polymer network and thereby stabilize the pericardial space;
extracting the cannula from the pericardial space, and
sealing of a transpericardial incision after extraction of the cannula.

12. The method of claim 11 further comprising deploying the constrainer components throughout the entire pericardial space.

13. The method of claim 11 further comprising constraining the heart with the polymer network.

14. The method of claim 13 further comprising constraining the heart to reduce a pumping load on the heart.

15. The method of claim 11 wherein induction of the constrainer includes applying radiation to the constrainer components by a radiation source projecting from a distal end of the cannula onto the constrainer components injected in the pericardial space.

16. A system for placing a heart constrainer in a patient comprising:
an injectable implant of a constrainer components in the form of solutions, suspensions, emulsions or gels;
a delivery system for the injectable components including a cannula having a distal end positionable in a pericardial space of the patient; and
an inducer operable to form a polymer network or matrix of the components in the pericardial space.

17. A system as in claim 16 wherein the constrainer components comprise a biopolymer.

18. A system as in claim 16 wherein the constrainer components comprise comments separately injected into the pericardial space.

19. A system as in claim 16 wherein the delivery system includes a first annular member having a first lumen, and a second annular member coupled to the first annular member having a second lumen, wherein collectively the first annular member and the second annular member have a diameter suitable for placement at a treatment site within a mammalian body.

20. An system as in claim 19 wherein a distal end of the first lumen is adjacent a distal end of the second lumen to allow a combining of treatment agents introduced through each of the first annular member and the second annular member.
